(19) United States
(12) Patent Application Publication
(10) Pub. No.: US 2019/0358127 A1
(43) Pub. Date: Nov. 28, 2019

(54) FINE NEEDLE DOSING ADAPTER

(71) Applicant: Scott WELLS, Old Westbury, NY (US)

(72) Inventor: Scott WELLS, Old Westbury, NY (US)

(21) Appl. No.: 16/397,232

(22) Filed: Apr. 29, 2019

Related U.S. Application Data
(60) Provisional application No. 62/674,944, filed on May 22, 2018.

(51) Int. Cl. A61J 1/20; A61M 5/178

(52) U.S. Cl. CPC A61J 1/2065; A61M 2205/0238; A61M 5/1782

(57) ABSTRACT

A fine needle dosing adaptor for dosing a fine needle syringe from a multi-dose vial has an adaptor body with an upper portion and a neck portion adapted to fit snugly within the neck of the multi-dose vial. A bore is centrally disposed within an upper portion of the adaptor body and has a diameter adapted to receive the hub of the fine needle syringe with a snug fit.

An inverted funnel-shaped opening is centrally disposed within the neck portion of the adaptor body and needle chamber extends between the bore and the inverted funnel-shaped opening. The diameter of the needle chamber is slightly larger than an outer diameter of the needle and the length of the needle chamber is shorter than a length of the needle.

### CROSS REFERENCE TO RELATED APPLICATIONS

This application claims priority under 35 U.S.C. § 119(e) and the benefit of co-pending U.S. Provisional Application No. 62/674,944 filed on May 22, 2018, the disclosure of which is hereby incorporated by reference in its entirety.

### BACKGROUND OF THE INVENTION

### Field of the Invention

This invention is intended for use in the dosing and administration of neurotoxin medications, such as for example, Botulinum toxin and/or any other injected medications which, because of their nature are injected trans-dermally via ultra-fine needles loaded from multidose vials

### The Prior Art

The standard method for reconstitution and dose preparation of currently available Botulinum toxins requires filling a pre-supplied tiny vial containing freeze-dried or anhydrous product with a small amount of liquid diluent (usually less than 3.0 cc). In doing so, a syringe carrying a relatively large bore needle (typically 18-22 gauge) is used to plunge through a 3-4 mm thick rubber stopper adding the required diluent. After reconstitution, dosing is usually accomplished by loading much smaller syringes (typically less than 0.3 cc) carrying integrated fine hollow bore needles, such as for example, insulin syringes (typically 30-31 gauge). An alternate method requires loading syringes with attachable fine gauge hollow-bore needles.

In either case, these tiny ultra-fine beveled point needles cannot be passed through the original stopper without destroying the integrity of the needle. Thus, the service provider or an assistant must remove the original stopper and undergo the rather awkward maneuver of dipping the dosing syringe with its attached needle through the rather small vial neck into a correct position to draw up a precise dose of product for administration.

This procedure usually requires an advanced amount of dexterity. Further complicating this maneuver is the added problem that if at any time the bevel point of the fine gauge needle should contact any part of the glass vial, even slightly, the delicate integrity of the needle is compromised. This causes risk of additional pain and the potential for cutaneous bruising in a patient, when blunted needles are used for percutaneous injection. Additionally complicating this loading maneuver, is a potential for microbial contamination of the liquid as syringes for this purpose are often vended in non-sterile, multiple unit packaging (i.e., only the needle and hub are sterile.)

Aside from the above practical need for an easier and safer way to load dose administration syringes is the issue of cost efficiency. All of the various neurotoxins are sold in multidose vials with a wholesale value of several hundred dollars. The waste of even a fractional drop of reconstituted product represents a significant value loss. This matter becomes more significant when trying to recover every drop of the last dose clinging to the inside of the bottle. The last dose is currently obtained by tilting the bottle on a steep incline driving the fluid into a corner of the bottom of the vial and then near certainly touching the fine needle to the glass corner to recover the last drop, as illustrated for example in FIG. 1.

Accordingly, there exists a need for a fine needle dosing adaptor for dosing a fine needle syringe from a multi-dose vial which overcomes these and other shortcomings of the heretofore known methods and arrangements.

### SUMMARY OF THE INVENTION

The invention relates to a fine needle dosing adaptor for dosing a fine needle syringe having a hub and a needle from a multi-dose vial having a neck.

A fine needle dosing adaptor according to an aspect of the invention includes an adaptor body with an upper portion bounded by an upper portion upper surface and an upper portion lower surface. The adaptor body further includes a neck portion extending from the upper portion lower surface to a neck portion lower surface. The neck portion of the adaptor body has an outside diameter adapted to fit snugly within the neck of the multi-dose vial. The upper portion of the adaptor body has an upper portion outside diameter larger than the neck portion outside diameter.

A bore is centrally disposed within the upper portion of the adaptor body and extends through the upper portion upper surface. The bore has a bore diameter adapted to receive the hub of the fine needle syringe with a snug fit.

An inverted funnel-shaped opening is centrally disposed within the neck portion of the adaptor body and extends through the neck portion lower surface.

A needle chamber extends between the bore and the inverted funnel-shaped opening. The diameter of the needle chamber is slightly larger than an outer diameter of the needle and the length of the needle chamber is shorter than a length of the needle.

According to a further aspect of the invention, the bore is substantially cylindrical.

According to a further aspect of the invention, the bore has a plurality of alignment slots adapted to engage a corresponding plurality of tabs provided on the hub of the fine needle syringe. The alignment slots may be two alignment slots arranged substantially parallel to one another. The alignment slots may have a height greater than a length of the needle.

According to a further aspect of the invention, the bore is adapted to form a water-tight seal with the hub of the fine needle syringe.

According to a further aspect of the invention, a surface of the neck portion defining the inverted funnel-shaped opening has a low surface tension surface, such as a low surface tension coating or a surface texturing.

An advantage of a fine needle dosing adaptor according to embodiments of the invention is that it allows for easy, rapid dosing into fine needle syringes without the worry of needle tip damage, nor product contamination. A fine needle dosing adaptor according to aspects of the invention is specifically designed to provide near absolute protection of the needle tip from bottle or adapter contact and is also designed to allow near total recovery of a reconstituted product.

### BRIEF DESCRIPTION OF THE DRAWINGS

Other benefits and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a conventional method of filling a fine needle syringe;

FIG. 2 shows a fine needle dosing adaptor according to an embodiment of the invention;

FIG. 3 shows a cross-sectional view of the fine needle dosing adaptor shown in FIG. 2;

FIG. 4 shows a cross-sectional view of the fine needle dosing adaptor shown in FIG. 2 and a multi-dose vial with a fine needle syringe inserted in the adaptor;

FIG. 5 shows a fine needle dosing adaptor according to another embodiment of the invention;

FIG. 6A shows a cap or stopper for the fine needle dosing adaptor shown in FIGS. 2-4; and

FIG. 6B shows a cap or stopper for the fine needle dosing adaptor shown in FIG. 5.

### DETAILED DESCRIPTION OF THE DRAWINGS

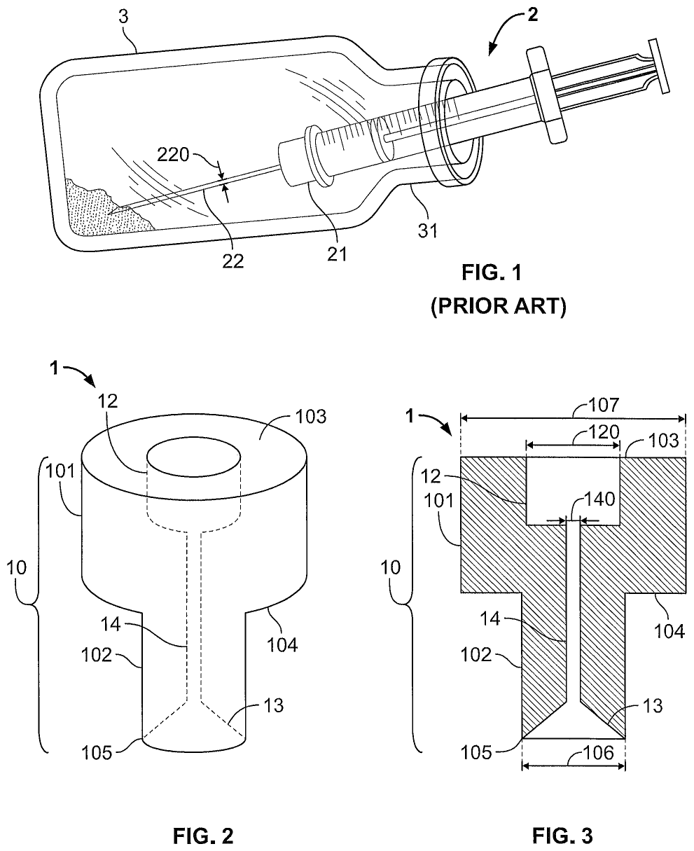

Referring now in detail to the drawings and, in particular, FIG. 1 shows a conventional method of filling a fine needle syringe 2 from a multi-dose vial 3 having a neck 31. The fine needle syringe has a hub or barrel portion 21 and a needle portion 22. The needle 22 has an outside diameter 220.

As shown, in the known method, a last dose is obtained by tilting the multi-dose vial or bottle 3 on a steep incline to drive the fluid into a corner of the bottom of the vial and then inserting the fine needle syringe 2 into the vial 3 at an angle, thereby with near certainty touching the fine needle to the glass corner to recover the last drop.

The proposed invention relates to a fine needle dosing adaptor which, when used to replace the original stopper of a multi-dose vial for a neurotoxin or other injected medication, allows for easy, rapid dosing into fine needle syringes without the risk of needle tip damage or product contamination. The inventive adaptor is very specifically designed to provide near absolute protection of the needle tip from bottle or adapter contact and is also designed to allow near total recovery of the reconstituted product.

As shown in FIG. 2, the adapter device 1 is roughly shaped like the original stopper along its outer dimensions. However, a central lumen is designed with unique properties to provide the needed advantages.

The fine needle dosing adaptor 1 includes an adaptor body 10. The adaptor body 10 may be made from rubber or any other suitable material. The adaptor body 10 has an upper portion 101 bounded by an upper portion upper surface 103 and an upper portion lower surface 104. The adaptor body 10 further includes a neck portion 102 extending from the upper portion lower surface 104 to a neck portion lower surface 105.

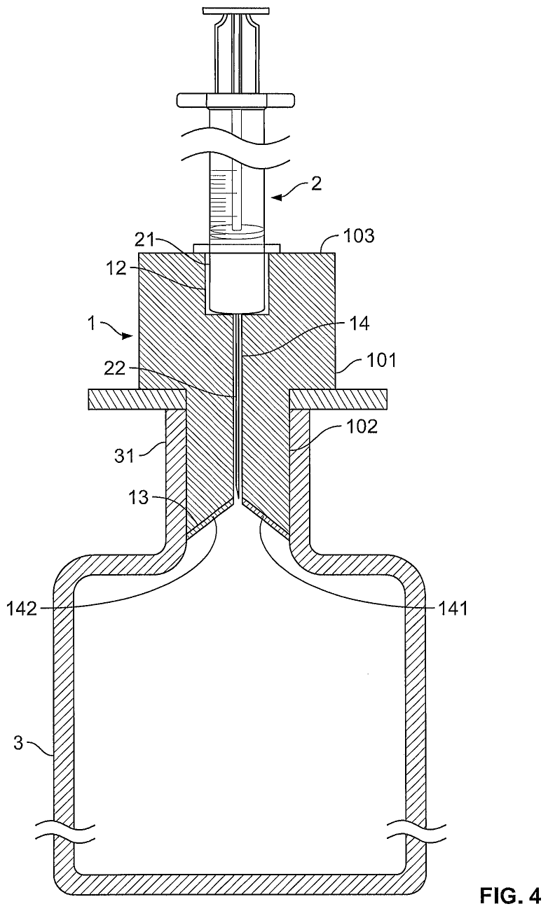

As shown in FIGS. 3 and 4, the neck portion 102 of the adaptor body 10 has an outside diameter 106 adapted to fit snugly within the neck 31 of the multi-dose vial 3. The upper portion 101 of the adaptor body 10 has an upper portion outside diameter 107 larger than the neck portion outside diameter 106.

At the top or proximal end of the adapter body 10 is a hollowed out bore 12 or hub chamber sized to allow the needle hub 21 of the syringe 2 to pressure fit into this orifice. The bore 12 is centrally disposed within the upper portion 101 of the adaptor body 10 and extends through the upper portion upper surface 103. As shown in FIGS. 3 and 4, the bore 12 has a bore diameter 120 adapted to receive the hub 21 of the fine needle syringe 2 with a snug fit.

An inverted funnel-shaped opening 13 is centrally disposed within the neck portion 102 of the adaptor body 10 and extends through the neck portion lower surface 105.

At the distal end of the bore or hub chamber 12 the lumen or opening continues as a narrow needle chamber 14 barely wider than the fine needle 22. This needle chamber 14 extends between the bore 12 and the inverted funnel-shaped opening 13. The diameter 140 of the needle chamber is slightly larger than an outer diameter 220 of the needle 22 and the length of the needle chamber is shorter than a length of the needle 221.

In use, as the syringe 2 and needle 22 are guided into the adaptor body 10 and associated opening, the tight fit of the bore 12 or hub chamber to the needle hub 21 guides the fine needle 22 down the needle chamber 14 parallel to its sides and avoiding any tip contact. The snug fit of the hub 21 into the rubber bore 12 or hub chamber further provides a sterile water tight seal preventing leakage when the multi-dose vial 3 is inverted for filling of the syringe 2. The bore 12 may also have a depth such that only the sterile hub 21 and not the non-sterile syringe barrel is received in the bore. There is, thus created, isolation of the non-sterile syringe barrel from the liquid as the sterile hub 21 is pressure fitted. Moreover, because the needle chamber 14 is so narrow, minimal loss of product occurs as the multi-dose vial 3 empties of its last remnants.

As shown, at its distal end the needle chamber 14 which is no longer than the fine needle 22, opens into an inverted funnel-shaped opening 13. This opening 13 may be lined with a very slick surface tension reducing surface. This low surface tension surface may be provided by a surface texturing 142 or an applied coating 141.

The portion of the device containing at least part of the needle chamber 14 and distal funnel 13 represents that portion of the adapter 1 which is inserted into the vial neck 31 in a manner similar to the original stopper. When the needle tipped syringe 2 is inserted properly into the adapter 1 and properly fitted into the multi-dose vial 3, the entire connected coupling of vial 3-adaptor 1-and syringe 2 is inverted to allow the plunger to simply withdraw the dose with greater ease. Once the dose is loaded the vial 3 is returned upright and the syringe 2 withdrawn. Any minor negative pressure in the vial 3 produced during withdrawal is now released, facilitating further dosing. In this manner, several syringes may be used with a single fine needle dosing adaptor 1 until every last drop of the valuable medication is safely and easily dosed. There is almost no waste because the vial 3 and the adapter 1 load every last drop down the slippery funnel shaped adapter into the tiny needle chamber.

In one embodiment, the fine needle adapter is designed to be used with existing fine needle syringes, such as widely used insulin syringes. As their needle hub is perfectly cylindrical in shape, the proximal aperture (bore 12) or needle hub is substantially cylindrical in shape to receive the cylindrical hub.

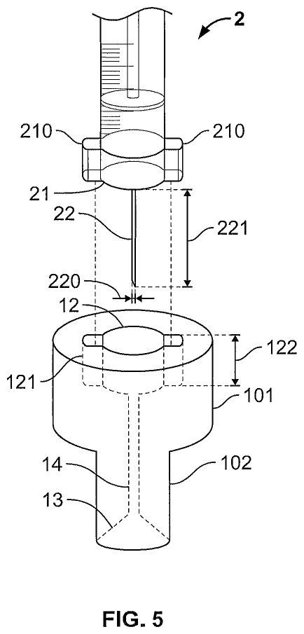

In a further embodiment, a new type of fine needle syringe would be created for use with a specifically designed fine needle dosing adaptor. In particular, with a cylindrical hub syringe there is still a chance that if the syringe 2 is tilted off a perpendicular trajectory into the adapter 1, the needle 22 may not be guided properly into the needle chamber 14. The further embodiment is adapted to be used with a needle hub having two or more small rectangular tabs 210 appearing like wings as illustrated in FIG. 5.

In this embodiment, the bore 12 of the fine needle dosing adaptor has a plurality of alignment slots 121 adapted to engage the corresponding plurality of tabs 210 provided on the hub of the fine needle syringe 2. The alignment slots may be two alignment slots 121 arranged substantially parallel to one another. The alignment slots 121 may have a height 122 greater than a length 221 of the needle.

The tabs 210 may be aligned at 180 degrees orientation along the axis of the syringe 2. The alignment slots 121 can be channeled into the needle hub chamber and arranged to accept the alignment tabs 210 provided on the syringe hub, thereby forcing an absolutely perpendicular trajectory of the needle 22 into the needle chamber 14. The fine needle syringe 2 may be designed so that the needle 22 itself is just slightly shorter than the height 122 of the alignment slots 121 or tabs 210. With these proportions, the tabs 210 engage the alignment slots 121 prior to the needle 22 entering the needle chamber 14, thereby preventing any tilting of the syringe axis off perpendicular. The needle 22 is thus absolutely guided into the needle chamber 14 with even less possibility of tip contact.

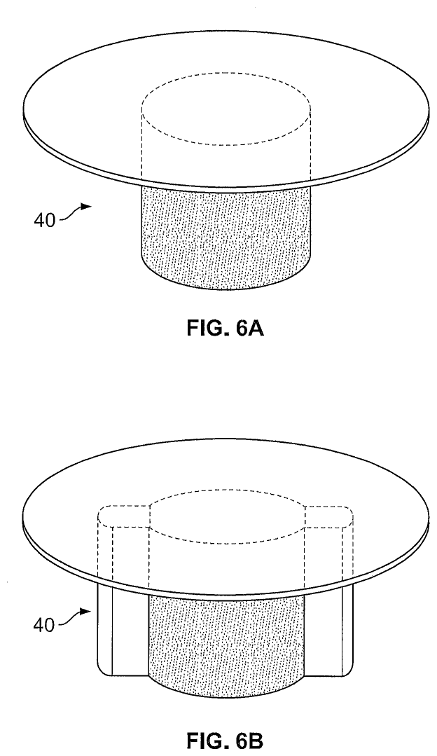

In the event that a partially emptied multi-dose vial 3 is to be stored for short time, a cap 40 may be placed on the top of the fine needle dosing adapter 1 fitting in to the bore 12 and sealing the vial 3 from air exposure or contamination. FIGS. 6A and 6B show caps or stoppers 40 for the different embodiments of the fine needle dosing adaptors according to the invention.

Although a fine needle dosing adapter disclosed herein is designed primarily for use with neurotoxin medications, it is adaptable to any small multi-dose medication vial.

Accordingly, while a number of embodiments of the present invention have been shown and described, it is obvious that many changes and modifications may be made thereunto without departing from the spirit and scope of the invention.

### LIST OF REFERENCE NUMERALS

1 fine needle dosing adaptor
2 fine needle syringe
3 multi-dose vial
10 adaptor body
12 bore
13 inverted funnel-shaped opening
14 needle chamber
21 hub
22 needle
31 neck
40 cap
101 upper portion
102 neck portion
103 upper portion upper surface
104 upper portion lower surface
105 neck portion lower surface
106 neck portion outside diameter
107 upper portion outside diameter
120 bore diameter
121 alignment slot
122 alignment slot height
140 needle chamber diameter
141 coating
142 texturing
210 alignment tab
220 needle outside diameter
221 needle length

What is claimed is:

1. A fine needle dosing adaptor for dosing a fine needle syringe having a hub and a needle from a multi-dose vial having a neck, the fine needle dosing adaptor comprising:
an adaptor body comprising:
an upper portion bounded by an upper portion upper surface and an upper portion lower surface;
a neck portion extending from said upper portion lower surface to a neck portion lower surface, said neck portion having a neck portion outside diameter adapted to fit snugly within the neck of the multi-dose vial and said upper portion having an upper portion outside diameter larger than said neck portion outside diameter;
a bore centrally disposed within said upper portion and extending through said upper portion upper surface, said bore having a bore diameter adapted to receive the hub of the fine needle syringe with a snug fit;
an inverted funnel-shaped opening centrally disposed within said neck portion and extending through said neck portion lower surface; and
a needle chamber extending between said bore and said inverted funnel-shaped opening, said needle chamber having a needle chamber diameter slightly larger than an outer diameter of the needle and a needle chamber length shorter than a length of the needle.

2. The fine needle dosing adaptor according to claim 1, wherein said bore is substantially cylindrical.

3. The fine needle dosing adaptor according to claim 1, wherein said bore has a plurality of alignment slots adapted to engage a corresponding plurality of tabs provided on the hub of the fine needle syringe.

4. The fine needle dosing adaptor according to claim 3, wherein said plurality of alignment slots comprise two alignment slots arranged substantially parallel to one another.

5. The fine needle dosing adaptor according to claim 3, wherein said plurality of alignment slots have a height greater than a length of the needle.

6. The fine needle dosing adaptor according to claim 1, wherein said bore is adapted to form a water-tight seal with the the hub of the fine needle syringe.

7. The fine needle dosing adaptor according to claim 1, wherein a surface of said neck portion defining said inverted funnel-shaped opening comprises a low surface tension surface.

8. The fine needle dosing adaptor according to claim 7, wherein said low surface tension surface comprises a coating.

9. The fine needle dosing adaptor according to claim 7, wherein said low surface tension surface comprises a surface texturing.
